(19) United States
(12) Patent Application Publication
(10) Pub. No.: US 2005/0027338 A1
(43) Pub. Date: Feb. 3, 2005

(54) Stretchable lead body, method of manufacture, and system

(75) Inventor: Roger John Hill, Richardson, TX (US)

Correspondence Address: DOCKET CLERK, DM/ANSI, P.O. BOX 802432, DALLAS, TX, 75380, US

(73) Assignee: ADVANCED NEUROMODULATION SYSTEMS, INC., Plano, TX (US)

(21) Appl. No.: 10/630,098

(22) Filed: Jul. 29, 2003

(51) Int. Cl. A61N 1/05

(52) U.S. Cl. 607/116

(57) ABSTRACT

An implantable lead body, and method of manufacturing same, and system for stimulation. The lead body includes an expanding/expansion section providing an additional length or slack in the lead body and allowing the lead body to expand or stretch with the body thereby reducing or eliminating the mechanical force or strain on the ends of the lead at the destination sites.

### TECHNICAL FIELD OF THE INVENTION

The present invention relates to electrical leads, and in particular, an electrical lead for use in the medical field.

### BACKGROUND

Implantable leads having electrodes are used in a variety of applications, including the delivery of electrical stimulation to surrounding tissue, neural or otherwise, as well as measuring electrical energy produced by such tissue. Some leads include lumens for the delivery of other elements, including chemicals and drugs. Whether in a stimulation, sensing or element delivery capacity, such leads are commonly implanted along peripheral nerves, within the epidural or intrathecal space of the spinal column, and around the heart, brain, or other organs or tissue of a patient.

Differing techniques have been utilized to construct or manufacture such leads. Some prior art leads and methods of manufacture have been disclosed in several United States patents, such as U.S. Pat. Nos. 5,016,646 (Gotthardt, et al.), 5,433,742 (Willis), 6,208,881 (Champeau) and 6,216,045 (Black, et al.), which are incorporated herein by reference.

Generally, several elements (conductors, electrodes and insulation) are combined to produce a lead body. A lead typically includes one or more conductors extending the length of the lead body from a distal end to a proximal end of the lead. The conductors electrically connect one or more electrodes at the distal end to one or more connectors at the proximal end of the lead. The electrodes are designed to form an electrical connection or stimulus point with tissue or organs. Lead connectors (sometimes referred to as contacts, or contact electrodes) are adapted to electrically and mechanically connect leads to implantable pulse generators or RF receivers (stimulation sources), or other medical devices. An insulating material typically forms the lead body and surrounds the conductors for electrical isolation between the conductors and protection from the external contact and compatibility with a body.

Such leads are typically implanted into a body at an insertion site and extend from the implant site to the stimulation site (area of placement of the electrodes). The implant site is typically a subcutaneous pocket that receives and houses the pulse generator or receiver (providing a stimulation source) The implant site is usually positioned a distance away from the stimulation site, such as near the buttocks or other place in the torso area. In most cases, the implant site (and insertion site) is located in the lower back area, and the lead may extend through the epidural space (or other space) in the spine to the stimulation site (middle or upper back, or neck or brain areas). Once the system is implanted, the system of leads and/or extensions may be subject to mechanical forces and movement in response to body movement. For example, when a patient bends over, or otherwise stretches the affected area, force is exerted on the lead in a general lengthwise direction (and laterally). This force may result in the end(s) of the lead moving within the body due to the rigidness of the lead and/or cause other problems at the implant site. Such a result is undesirable.

In an effort to alleviate this problem, service loops are sometimes used. A service loop is an extra length of lead implanted in the body that provides an additional length when needed (i.e., looped). However, fibrous or scar tissue may grow and build up around the loop tending the loop to act as an anchor, thus failing to allow an increase in the length of the lead when desired.

Accordingly, there exists a need for a lead that includes an expandable/expansion section that stretches to accommodate motion in the body, results in less mechanical strain on the site of the implant, or on the implanted lead (distal and proximal ends of the lead), and further allows the use of conventional surgical tools and techniques for implant of the lead into the body.

### SUMMARY

According to the present invention, there is provided a lead. The lead includes a lead body having a proximal end and a distal end, and at least one expanding/expansion section. A connector and electrode are positioned proximate the respective ends of the lead body with a conductor extending through the lead body and electrically connecting the connector and the electrode. In another embodiment, the lead body has at least one section including means for expanding.

In another embodiment of the present invention, there is provided a method of manufacturing a lead. The method includes providing a lead body having a first diameter and a proximal end and a distal end with the lead body having at least one conductor extending through the lead body. The method further includes forming an expanding/expansion section in the lead body.

In yet another embodiment of the present invention, there is provided a system for stimulating a portion of a body. The system includes a source for generating a stimulus and a lead. The lead includes a lead body having a proximal end and a distal end, and at least one expanding/expansion section. A connector and electrode are positioned proximate the respective ends of the lead body with a conductor extending through the lead body and electrically connecting the connector and the electrode. In another embodiment, the lead body has at least one section including means for expanding.

### BRIEF DESCRIPTION OF THE DRAWINGS

For a more complete understanding of the present invention, and the advantages thereof, reference is now made to the following descriptions taken in conjunction with the accompanying drawings, wherein like numbers designate like objects, and in which:

FIG. 1 is perspective view of a lead in accordance with the present invention;

FIG. 2 is a partial and more detailed view of the lead body shown in FIG. 1;

FIG. 3 is perspective view of a lead in accordance with the present invention;

FIG. 4 illustrates one embodiment of a system for stimulation in accordance with the present invention; and

FIG. 5 illustrates another embodiment of a system for stimulation in accordance with the present invention.

### DETAILED DESCRIPTION OF THE INVENTION

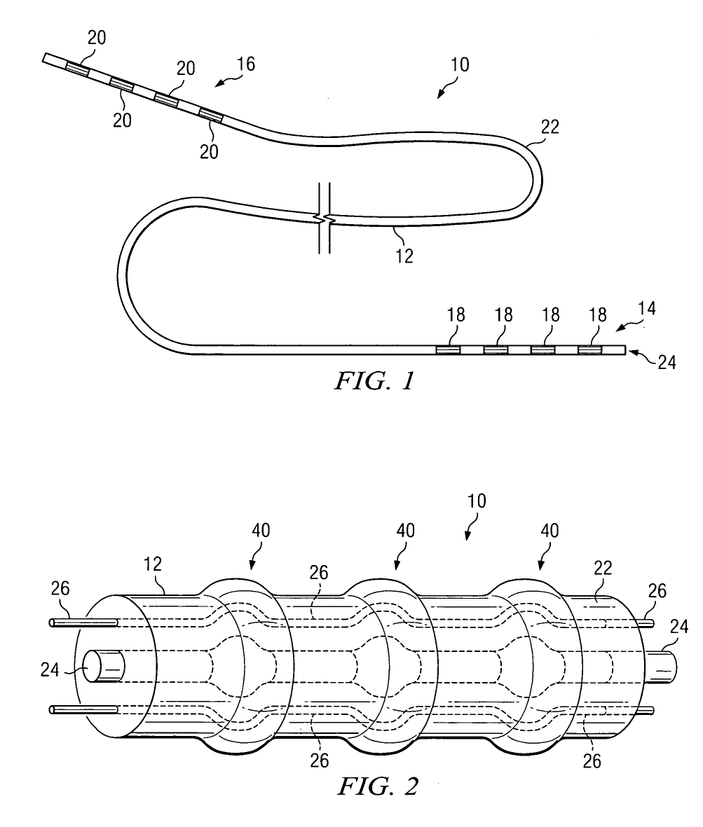

With reference to FIG. 1, there is illustrated an embodiment of a lead 10 in accordance with the present invention. The lead 10 includes a distal end 14 and a proximal end 16. The lead 10 includes a lead body 12 that extends from the distal end 14 to the proximal end 16. The distal end 14 of the lead 10 is shown including four band electrodes 18. The proximal end 16 of the lead 10 is shown including four contact electrodes (or ring electrodes) 20 that form a lead connector. The lead 10 generally includes one or more conductors 26 (see FIG. 2) extending a substantial portion of the lead 10 to electrically connect the contact electrodes 20 to respective band electrodes 18. An optional lumen 24 is shown that extends through the lead 10 and may be used for different purposes, including the delivery of chemicals or drugs.

As will be appreciated, any number of conductors 26, electrodes 18 and contact electrodes 20 may be utilized, as desired. For purposes of illustration only, the lead 10 is shown with four contact electrodes 20 and four electrodes 18. It will be further understood that the distal end 14 of the lead 10 is shown with band electrodes 18. Other types, configurations and shapes of electrodes may be used, including percutaneous, paddle-shaped, and the like, etc. as known to those skilled in the art. Likewise, other types, configurations and shapes of contact electrodes (and lead connectors) may be used, as desired.

Typically, the lead body 12 is a structure having a round cross-section. Alternatively, the cross-section of the lead body 12 may be configured in any number of cross-sectional shapes appropriate for the specific application. The figures and following description generally refer to a round cross-sectional shape for the lead body 12 for illustrative purposes only. The lead body generally includes a lead body insulator 22 configured to insulate the conductors 26 and presents a biocompatible external surface to the body tissue. In one embodiment, the lead body insulator 22 is coextensive with the conductors 26.

The lead body insulator 22 is formed of insulating material typically selected based upon biocompatibility, biostability and durability for the particular application. The insulator material may be silicone, polyurethane, polyethylene, polyamide, polyvinylchloride, PTFT, EFTE, or other suitable materials known to those skilled in the art. Alloys or blends of these materials may also be formulated to control the relative flexibility, torqueability, and pushability of the lead 10. Depending on the particular application, the diameter of the lead body 12 may be any size, though a smaller size is more desirable for neurological and myocardial mapping/ablation leads and neuromodulation and stimulation leads.

The conductors 26 may take the form of solid wires, drawn-filled-tube (DFT), drawn-brazed-strand (DBS), stranded wires or cables, ribbons conductors, or other forms known or recognized to those skilled in the art. The composition of the conductors 26 may include aluminum, stainless steel, MP35N, platinum, gold, silver, copper, vanadium, alloys, or other conductive materials or metals known to those of ordinary skill in the art. The number, size, and composition of the conductors 26 will depend on the particular application for the lead 10, as well as the number of electrodes.

The conductors 26 may be configured along the lead body 12 in a straight orientation or spirally or helically wound about the lumen 24 or center of the lead body 12. The conductors 26 are typically insulated from the lumen 24, from each other, and from the external surface of the lead 10 by the insulative material 22. The insulative material 22 may be of a single composition, or multiple layers of the same or different materials.

At least one electrode 18 is positioned at the distal end 14 of the lead body 12 for electrically engaging a target tissue or organ. In addition, at least one connector 20 is positioned at the proximal end 16 of the lead body 12 for electrically connecting the conductors 26 to a stimulating or receiving source. In one embodiment, the lead 10 is generally configured to transmit an electric signal from an electrical source (see FIGS. 4 and 5) for application at, or proximate to, a spinal nerve or peripheral nerve.

The electrodes 18 and contact electrodes 20 are typically made of a conductive material such as platinum, gold, silver, platinum-iridium, stainless steel, MS35N, or other conductive materials, metals or alloys known to those skilled in the art. The size of the electrodes 18 are generally chosen based upon the desired application. The contact electrodes 20 generally have a size and configuration appropriate to connect the lead 10 to a desired electrical source or receiver.

With reference to FIG. 2, there is illustrated a detailed perspective view of a section of the lead body 12 of the present invention. The lead body 12 includes expanding (or expansion) sections 40 or bubbles, and may also be described as a deformation with respect to the original configuration of the lead body 12. The sections 40 surround at least a portion of the lead body 12, and in one embodiment are shown encompassing the entire diameter of the lead body 12. The section 40 provides a means for adding (or providing) length or slack for the lead 10 and associated conductors 26 which, in one embodiment, substantially conform to the bubble shape of expansion section 40 of the lead body 12. This means allows the lead 10 to stretch lengthwise (or longitudinally/axially) or bend without substantial movement of the distal end 14 in relation to a fixed location along the lead body 12, and/or reduces the force on the conductors 26 (and lead body 12) having orientation lengthwise, thus the expansion section 40 provides increased elasticity of the lead body 12. As will be appreciated, leads having conductors oriented substantially lengthwise (or substantially parallel) to the lead body will likely benefit more from the present invention, as opposed to leads without such an expansion section that rely on helically or spirally wound conductors 26 to allow for longitudinal lead tension.

Generally, an implanted lead 10 is configured to be fixed or stationary at a specific location near the implant site. It is advantageous for the distal end 14 to remain substantially fixed relative to the desired location of the electrodes 18 when implanted. Further, it is desirable to reduce any mechanical force that may occur at the proximal end 16. As such, when human body implanted with the lead 10 bends or moves, the sections 40 provide additional length or slack, which reduces the amount of tension or force on the fixed locations, thus reducing movement of the lead 10 within the body and helping to maintain the position of the electrodes 18 within the body. This also reduces mechanical strain on the lead connector(s) (and contact electrodes) at the site of the implant. In other words, the lead 10 including the sections 40 allow the lead 10 to “stretch” when the body moves or stretches while reducing the mechanical force or strain at the distal end 14 and proximal end 16 of the lead 10.

The expanding (or expansion) sections 40 are longitudinally spaced along the lead body 12. In one embodiment, each section 40 runs circumferentially around the lead body 12, in the form of a ring. Any number of sections 40 may be utilized along the lead body 12, as desired consistent with the present invention. In one embodiment, the sections are spaced apart about one-quarter of an inch (i.e., roughly 4 sections/inch). The number and positioning of the sections 40 along the lead body 12, and spacing therealong, will depend, at least in part on the desired length of the lead 10, including the length implanted within the body, and the intended or anticipated movement of the body. The sections 40 allow the lead 10 as a whole to stretch or expand longitudinally, and also allow the conductors 26 embedded within the lead body 12 to stretch or expand as well. As shown, the conductors 26 (shown as dotted lines) at the locations of the sections 40 are also similar in shape to the sections (i.e., bubble-shaped or curved outwardly), or conformal to or within the expansion ring or section 40. In addition, the optional lumen 24 is similarly shaped.

The expansion sections 40 protrude from the lead body 12 in relation to the outside diameter of the lead body 12 (as compared to a typical prior art lead) The size (height and length) of the sections 40 are generally of a size and configuration appropriate to advantageously reduce or eliminate the mechanical force or strain at the ends when the body moves. In one embodiment, the height of the sections (relative to the surrounding diameter of the lead body 12) is about 0.05 inches. In another embodiment, the height is about 0.1 inches. In yet another embodiment, the diameter of the section 40 is at least about twice the diameter of the lead body.

The sections 40 are shown shaped as a bubble or curved outwardly. Different shapes may be utilized sufficient to provide extra length or slack in the lead body 12 as described above.

As the lead body 12 is bent or stretched, the sections 40 expand, at least in part longitudinally, and therefore the lead 10 similarly expands. The expanding (or expandable) sections 40 provide an expanding (or stretchable or elastic) mechanism for the lead body 12 (and for the conductors 26 embedded therein).

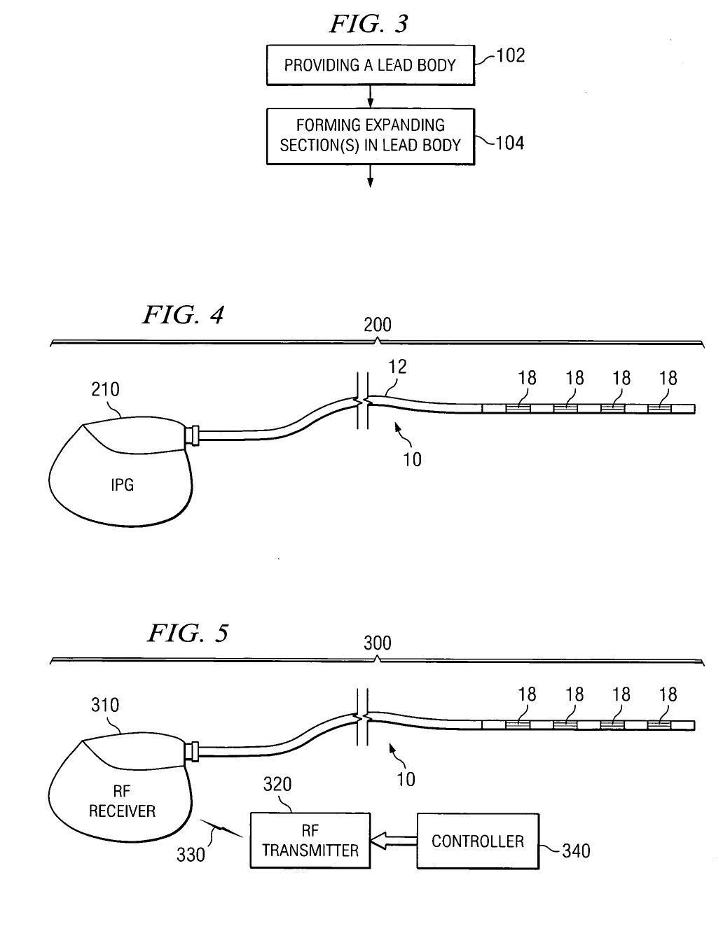

With reference to FIG. 3, there is shown a diagram of a process for manufacturing the lead 10 of the present invention. At a step 100, a typical lead body is provided. The lead body may be a lead body constructed according to methods typically available or known to those skilled in the art. At a step 102, one or more expansion sections 40 are formed on the lead body 12 (see FIG. 2).

In one embodiment, to form the sections 40 in the step 102, one or more selected portions of the lead body 12 are heated to a predetermined temperature to generate the sections.

In another embodiment, to form the sections 40 in the step 102, one or more selected portions of the lead body 12 are heated to a predetermined temperature and pressure is applied in a longitudinal/axial direction (from one side or both sides) to the heated portion. In other words, the lead body 12 is compressed longitudinally/axially at the heated portion to create the section 40. The pressure is applied during the heating, or shortly thereafter, when the portion is still deformable or malleable due to the increase in temperature.

As will be appreciated, a typical lead body 12 may be purchased from a vendor and the expanding sections 40 formed. Alternatively, during construction of a typical lead body, the process may be altered which includes one or more of the steps recited above to generate the sections 40.

It will be understood that the temperature to which the selected portion(s) (or insulative material 22) of the lead body 12 are heated depends on the desired size and form of the section 40 and the composition of the material utilized for the insulative body material 22. For example, and for illustrative purposes only, in one embodiment, the lead body 12 is constructed of an insulative material, and more particularly constructed of polyurethane, and the predetermined temperature should range from about 120 to 150 degrees Celsius. It will be understood that utilization of different composition(s) coupled with the desired size and configuration of the expanding section(s) may require different predetermined temperatures in order to generate the desired sections 40.

In one embodiment, a heating element (not shown) is positioned at or near the selected portion(s) of the lead body 12 to generate heat applied to the body 12 and produce the desired section(s) 40. The heating element may be any size and shape, but in one embodiment, the element is arcuate shaped, and surrounds either a portion of the lead body 12 or the entire lead body 12. In general terms, the heating of a portion of the lead body 12 expands the insulative material 22 to form the sections 40. In the other embodiment including the step of applying pressure to compress the lead body 12, any means or method known to those skilled in the art may be utilized to apply the pressure.

Now referring to FIGS. 4 and 5, there are shown two embodiments of a stimulation system 200, 300 in accordance with the present invention. The stimulation systems generate and apply a stimulus to a tissue or to a certain location of a body. In general terms, the system 200, 300 includes a stimulation or energy source 210, 310 and a lead 10 for application of the stimulus. The lead 10 shown in FIGS. 4 and 5 is the lead 10 of the present invention.

As shown in FIG. 4, the stimulation system 200 includes the lead 10 that is coupled to the stimulation source 210. In one embodiment, the stimulation source 210 includes an implantable pulse generator (IPG). As is known in the art, an implantable pulse generator (IPG) is capable of being implanted within the body (not shown) that is to receive electrical stimulation from the stimulation source 210. An exemplary IPG may be one manufactured by Advanced Neuromodulation Systems, Inc., such as the Genesis® System, part numbers 3604, 3608, 3609, and 3644.

As shown in FIG. 5, the stimulation system 300 includes the lead 10 that is coupled to the stimulation source 310. The stimulation source 310 includes a wireless receiver (not shown). The stimulation source 310 may also be referred to as a wireless receiver. As is known in the art, the stimulation source 310 comprising a wireless receiver is capable of being implanted within the body (not shown) that is to receive electrical stimulation from the wireless receiver 310. An exemplary wireless receiver 310 may be those receivers manufactured by Advanced Neuromodulation Systems, Inc., such as the Renew® System, part numbers 3408 and 3416.

The wireless receiver (not shown) within stimulation source 310 is capable of receiving wireless signals from a wireless transmitter 320. The wireless signals are represented in FIG. 5 by wireless link symbol 330. The wireless transmitter 320 and a controller 340 are located outside of the body that is to receive electrical stimulation from the stimulation source 310. A user of the stimulation source 310 may use the controller 340 to provide control signals for the operation of the stimulation source 310. The controller 340 provides control signals to the wireless transmitter 320. The wireless transmitter 320 transmits the control signals (and power) to the receiver in the stimulation source 310, and the stimulation source 310 uses the control signals to vary the signal parameters of the electrical signals that are transmitted through lead 10 to the stimulation site. An exemplary wireless transmitter 320 may be those transmitters manufactured by Advanced Neuromodulation Systems, Inc., such as the Renew® System, part numbers 3508 and 3516.

As will be appreciated, the contact electrodes 20 are not visible in FIG. 4 (or FIG. 5) because the contact electrodes 20 are situated within a receptacle (not shown) of the stimulation source 210, 310. The contact electrodes 20 are in electrical contact with a generator (not shown) of electrical signals within the stimulation source 210, 310. The stimulation source 210, 310 generates and sends electrical signals via the lead 10 to the electrodes 18. Understandably, the electrodes 18 are located at a stimulation site (not shown) within the body that is to receive electrical stimulation from the electrical signals. A stimulation site may be, for example, adjacent to one or more nerves in the central nervous system (e.g., spinal cord). The stimulation source 210, 310 is capable of controlling the electrical signals by varying signal parameters (e.g., intensity, duration, frequency) in response to control signals that are provided to the stimulation source 210, 310.

It may be advantageous to set forth definitions of certain words and phrases that may be used within this patent document: the terms “include” and “comprise,” as well as derivatives thereof, mean inclusion without limitation; the term “or,” is inclusive, meaning and/or; the phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like; and if the term “controller” is utilized herein, it means any device, system or part thereof that controls at least one operation, such a device may be implemented in hardware, firmware or software, or some combination of at least two of the same. It should be noted that the functionality associated with any particular controller may be centralized or distributed, whether locally or remotely.

Although the present invention and its advantages have been described in the foregoing detailed description and illustrated in the accompanying drawings, it will be understood by those skilled in the art that the invention is not limited to the embodiment(s) disclosed but is capable of numerous rearrangements, substitutions and modifications without departing from the spirit and scope of the invention as defined by the appended claims.

1. A lead, comprising:
a lead body having a proximal end and a distal end, and the lead body having at least one expansion section;
at least one connector positioned proximate the proximal end;
at least one electrode positioned proximate the distal end; and
at least one conductor extending through the lead body and electrically connecting the connector and the electrode.

2. The lead in accordance with claim 1 wherein the expansion section is formed by heating at least a portion of the lead body.

3. The lead in accordance with claim 1 wherein a diameter of the expansion section is greater than a diameter of an adjacent portion of the lead body.

4. The lead in accordance with claim 3 wherein the diameter of the expansion section is at least about two times greater than the diameter of the adjacent portion.

5. The lead in accordance with claim 1 wherein the lead body comprises a plurality of expansion-sections and two adjacent expansion sections are spaced apart less than one inch.

6. The lead in accordance with claim 1 wherein the expansion section forms an expansion ring around the lead body.

7. The lead in accordance with claim 6 wherein the at least one conductor is substantially conformal within the expansion ring.

8. The lead in accordance with claim 1 wherein the expansion section functions to allow expansion of the lead body in a longitudinal direction.

9. The lead in accordance with claim 9 wherein the expansion section provides increased elasticity of the lead body.

10. The lead in accordance with claim 1 wherein the expansion section is bubble-shaped.

11. A lead, comprising:
a lead body having a proximal end and a distal end, and the lead body having at least one section comprising means for expanding;
at least one connector positioned proximate the proximal end;
at least one electrode positioned proximate the distal end; and
at least one conductor extending through the lead body and electrically connecting the connector and the electrode.

12. The lead in accordance with claim 11 wherein the means for expanding comprises an expansion section wherein a diameter of the expansion section is greater than a diameter of an adjacent portion of the lead body.

13. The lead in accordance with claim 11 wherein the means for expanding comprises a plurality of expansion sections.

14. The lead in accordance with claim 11 wherein the means for expanding functions to allow expansion of the lead body in a longitudinal direction.

15. A method of manufacturing a lead, comprising:
providing a lead body having a first diameter and a proximal end and a distal end, the lead body having at least one conductor extending through the lead body; and
forming at least one expansion section within the lead body.

16. The method in accordance with claim 15 wherein a portion of the expansion section has a second diameter, the first diameter less than the second diameter.

17. The method in accordance with claim 15 wherein forming the at least one expansion section comprises:
heating at least a portion of the lead body to a predetermined temperature to form the expansion section.

18. The method in accordance with claim 17 wherein a portion of the expansion section has a second diameter, the first diameter less than the second diameter.

19. The method in accordance with claim 17 further comprising:
compressing the heated portion of the lead body to form the expansion section.

20. The method in accordance with claim 1 wherein a portion of the expansion section has a second diameter, the first diameter less than the second diameter.

21. A lead manufactured in accordance with the method as defined in claim 15.

22. A system for stimulating a portion of a body, the system comprising:
a source for generating a stimulus; and
an implantable lead for receiving the stimulus from the source, the implantable lead comprising,
a lead body having a proximal end and a distal end, and the lead body having at least one expanding section,
at least one connector positioned proximate the proximal end,
at least one electrode positioned proximate the distal end, and
at least one conductor extending through the lead body and electrically connecting the connector and the electrode.

23. The system in accordance with claim 22 further comprising:
a controller operable for communicating with the source and controlling the source.

24. The system in accordance with claim 22 wherein the source comprises an RF receiver.

25. The system in accordance with claim 22 wherein the source comprises an implantable pulse generator.

26. A system for stimulating a portion of a body, the system comprising:
a source for generating a stimulus; and an implantable lead for receiving the stimulus from the source, the implantable lead comprising,
a lead body having a proximal end and a distal end, and the lead body having at least one section comprising means for expanding,
at least one connector positioned proximate the proximal end,
at least one electrode positioned proximate the distal end, and
at least one conductor extending through the lead body and electrically connecting the connector and the electrode.
